(19) United States
(12) Patent Application Publication
(10) Pub. No.: US 2017/0137419 A1
(43) Pub. Date: May 18, 2017

(54) DERIVATIVES OF 2H-PYRAZOLO[4,3-C]QUINOLIN-3(5H)-ONE AND USE THEREOF

(71) Applicants: UNIVERSITE DU DROIT ET DE LA SANTE LILLE 2, Lille (FR); INSERM (INSTITUT NATIONAL DE LA SANTE ET DE LA RECHERCHE MEDICALE), Paris (FR); CENTRE HOSPITALIER REGIONAL ET UNIVERSITAIRE DE LILLE (CHRU), Lille (FR)

(72) Inventors: Régis Millet, Harnes (FR); Philippe Chavatte, Gondecourt (FR); Pierre Desreumaux, Mouvaux (FR); Mathilde Body, Phalempin (FR); Jamal El Bakali, Lille (FR)

(21) Appl. No.: 15/317,827

(22) PCT Filed: Jun. 11, 2015
(86) PCT No.: PCT/FR2015/051549

§ 371 Date: Dec. 9, 2016

(30) Foreign Application Priority Data
Jun. 12, 2014 (FR) 14 55322

(51) Int. Cl. C07D 471/04

(52) U.S. Cl. CPC C07D 471/04

(57) ABSTRACT

The invention relates to compounds of formula (I) or the pharmaceutically acceptable solvates thereof, as well as to the use thereof as a drug.

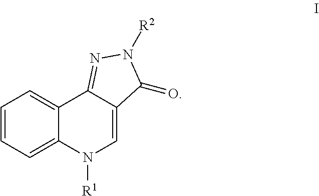

The present invention relates to novel derivatives of 2H-pyrazolo[4,3-c]quinolin-3(5H)-one of formula I and use thereof in the treatment and/or prevention of illnesses or diseases associated with a change (increase or decrease) in the activity of the CB₂ receptor, in particular autoimmune illnesses.

### PRIOR ART

The CB₂ receptor is mainly expressed in the cells associated with the immune system, such as leucocytes, macrophages, and B and T lymphocytes. This receptor is also present in the spleen, the tonsils and the prostate, and on the glial cells (microglia, astrocytes).

The location of the CB₂ receptor at the immune cells reveals the involvement of this receptor in the immune-modulating effects caused by cannabinoids. The CB₂ cannabinoid receptor appears to be an important mediator in the release of inflammatory cytokines and several in vitro and in vivo studies emphasise that the ligands of the CB₂ receptors constitute potential therapeutic agents for illnesses or diseases associated with a change (increase or decrease) in the activity of the CB₂ receptor, in particular autoimmune illnesses causing chronic inflammation. As the for CB₁ receptor, this is responsible for the psychotropic effects caused by cannabinoids and in particular by THC (Δ-9-tetrahydrocannabinol), the main psychoactive component found in cannabis. Thus the location of the CB₂ receptor suggests its important role in the control of the homeostasis of the immune system (production of pro- and anti-inflammatory cytokines, migration, proliferation and activation of immune cells) [Recent advances in the development of selective CB(2) agonists as promising anti-inflammatory agents. Leleu-Chavain N, Body-Malapel M, Spencer J, Chavatte P, Desreumaux P, Millet R. Curr Med Chem. 2012; 19 (21):3457-74.]. The absence of immunomodulation caused by cannabinoids in mice with no CB₂ receptors emphasises the role of CB₂ in many illnesses such as inflammatory illnesses, such as neurodegenerative diseases (MS, Alzheimer's disease, Parkinson's disease, and neurocognitive disorders associated with HIV-1) or chronic inflammatory bowel diseases (CIBD), but also atherosclerosis, pain, cancer, osteoporosis or liver diseases for example [From cannabis to selective CB2R agonists: molecules with numerous therapeutical virtues. Leleu-Chavain N, Biot C, Chavatte P, Millet R. Med Sci (Paris). 2013 May; 29 (5):523-8].

A large number of agonists of CB₂ currently known are not selective vis-à-vis the CB₁ receptor and therefore have undesirable secondary effects in the central nervous system, such as euphoria, sedation, hypothermia and catalepsy.

The patent application WO 2010/133973 A1 describes derivatives of 1,4-dihydropyridine and use thereof as a modulator of the CB₂ receptor. Some of these compounds are in fact selective agonists of CB₂.

Manera et al. (Bioorg. Med. Chem. Lett. 17 (2007) 6505-6510) describe 2H-pyrazolo[4,3-c]quinolin-3(5H)-ones. These compounds do however show a weak affinity for the CB1 and CB₂ receptors.

The patent application US 2012/0196845 describes 2H-pyrazolo[4,3-c]quinolin-3(5H)-ones and use thereof as positive allosteric modulators of the M1 receptor, in particular in the treatment of Alzheimer's disease.

There does however still exist a need for novel agonists of CB₂ that are selective vis-à-vis CB₁.

### SUMMARY OF THE INVENTION

The inventors have now succeeded in developing novel agonists of CB₂ that are selective vis-à-vis CB₁. These agonists have the advantage of not having the undesirable secondary effects associated with the activation of the CB₁ receptor, such as euphoria, sedation, hypothermia and catalepsy.

The invention therefore relates to compounds of formula I, the pharmaceutically acceptable solvates thereof and the use of these compounds, or their solvates or compositions containing them, as agonists of CB₂.

In a first aspect, the invention relates to compounds of formula I:

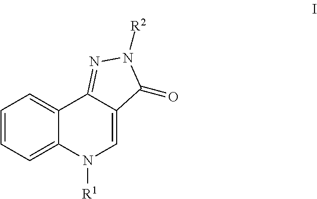

where or the pharmaceutically acceptable solvates thereof,
in which
R¹ is a linear C4 to C6 alkyl, linear C4 to C6 haloalkyl or C1 to C2 tetrahydropyranylalkyl; and
R² is C3 to C6 alkyl, cycloalkyl or cycloalkylalkyl.

In another aspect, the invention relates to pharmaceutical compositions comprising at least one compound according to the invention or one of the pharmaceutically acceptable solvates thereof and at least one pharmaceutically acceptable excipient.

As indicated above, the invention also relates to the use of the compounds according to the invention or one of the pharmaceutically solvates thereof as agonists of CB₂. Consequently the compounds of the invention and the pharmaceutically acceptable solvates thereof are useful in the treatment and/or prevention of illnesses or diseases associated with a change (increase or decrease) in the activity of the CB₂ receptor. The invention therefore also relates to the compounds according to the invention for use as a drug, in particular in the treatment and/or prevention of illnesses or diseases mediated by CB₂.

### DETAILED DESCRIPTION OF THE INVENTION

As detailed above, the invention relates to compounds of Formula I as well as the pharmaceutically acceptable solvates thereof.

Preferred compounds of Formula I and pharmaceutically acceptable solvates thereof are those in which R¹ and/or R² are defined as follows:

R¹ is linear C4 to C6 alkyl, linear C3 to C5 trifluoromethylalkyl or C1 to C2 tetrahydropyranylalkyl; preferably R¹ is linear C5 to C6 alkyl (n-pentyl and n-hexyl), linear C3 trifluoromethylalkyl (1,1,1-trifluoro-n-butyl) or C1 tetrahydropyranylalkyl (tetrahydropyranylmethyl); or R¹ is linear C5 alkyl (n-pentyl), linear C3 trifluoromethylalkyl (1,1,1-trifluoro-n-butyl) or C1 to C2 tetrahydropyranylalkyl, more preferentially R¹ is linear C5 alkyl (n-pentyl);

R² is C5 to C6 alkyl, C5 to C12 cycloalkyl or C3 to C12 cycloalkyl—C1 to C3 linear alkyl, preferably R² is n-pentyl, n-hexyl, C5 to C12 cycloalkyl or C3 to C12 cycloalkyl-methyl or C3 to C12 cycloalkyl-ethyl; more preferentially R² is n-pentyl, n-hexyl, C5 to C10 cycloalkyl or C6 to C10-methyl cycloalkyl or C6 to C10 cycloalkyl-ethyl, more preferentially still R² is n-pentyl, n-hexyl, cyclohexyl, cyclopropylmethyl, cyclohexylmethyl, cyclohexylethyl, adamantylmethyl, in particular adamant-1-ylmethyl or adamantylethyl, in particular adamant-1-ylethyl, more preferentially still R² is adamantylmethyl or adamantylethyl, in particular adamant-1-ylmethyl and adamant-1-ylethyl, and entirely preferably R² is adamantylmethyl, in particular adamant-1-ylmethyl.

In a particular embodiment, the compounds of Formula I and the pharmaceutically acceptable solvates thereof are those in which R¹ is as defined above and R² is chosen from the group consisting of n-pentyl, n-hexyl, cyclohexyl, cyclopropylmethyl, cyclohexylmethyl, cyclohexylethyl, adamantylmethyl and adamantylethyl, preferably R² is chosen from the group consisting of n-pentyl, n-hexyl, cyclohexyl, cyclopropylmethyl, cyclohexylmethyl, cyclohexylethyl, adamant-1-ylmethyl and adamant-1-ylethyl.

In fact, and without wishing to be bound by a theory, the inventors think that the selectivity of the compounds of the invention for CB₂ versus CB₁ is obtained by means of the bulky hydrophobic R² group. At the same time, this group allows good affinity of the compounds of the invention for CB₂. Advantageously, the compounds of the invention have an affinity for the CB₂ receptor of around one nanomole, in particular less than 100 nM, preferably less than 50 nM and more preferentially still less than 30 nM.

Particular preferred compounds of the invention are those listed in Table 1 below:

TABLE 1

| Structures | Name |
|---|---|
| 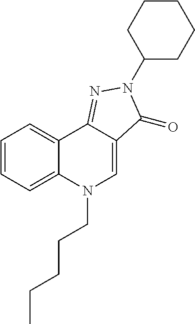 [SMILES: CCCCCn1cc2c(=O)n(C3CCCCC3)nc-2c2ccccc21] | 2-Cyclohexyl-5-pentyl-2H-pyrazolo[4,3-c]quinolin-3(5H)- one; |
| 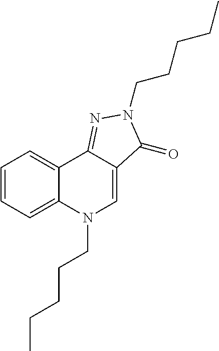 [SMILES: CCCCCn1nc2c3ccccc3n(CCCCC)cc-2c1=O] | 2,5-Dipentyl-2H-pyrazolo[4,3-c]quinolin-3(5H)-one; |
| 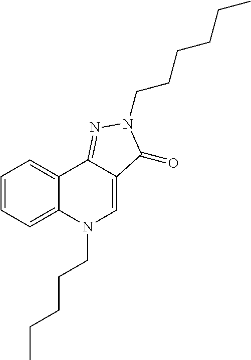 [SMILES: CCCCCCn1nc2c3ccccc3n(CCCCC)cc-2c1=O] | 2-Hexyl-5-pentyl-2H-pyrazolo[4,3-c]quinolin-3(5H)-one; |
| 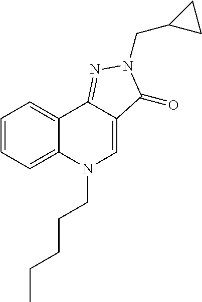 [SMILES: CCCCCn1cc2c(=O)n(CC3CC3)nc-2c2ccccc21] | 2-(Cyclopropylmethyl)-5-pentyl-2H-pyrazolo[4,3-c]quinolin- 3(5H)-one; |
| 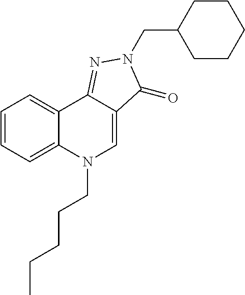 [SMILES: CCCCCn1cc2c(=O)n(CC3CCCCC3)nc-2c2ccccc21] | 2-(Cyclohexylmethyl)-5-pentyl-2H-pyrazolo[4,3-c]quinolin- 3(5H)-one; |
| 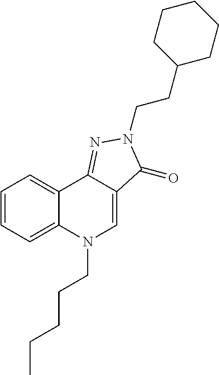 [SMILES: CCCCCn1cc2c(=O)n(CCC3CCCCC3)nc-2c2ccccc21] | 2-(Cyclohexylethyl)-5-pentyl-2H-pyrazolo[4,3-c]quinolin- 3(5H)-one; |
| 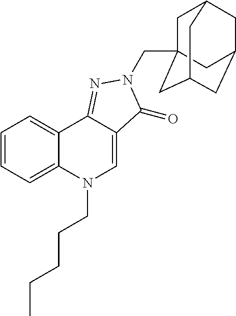 [SMILES: CCCCCn1cc2c(=O)n(CC34CC5CC(CC(C5)C3)C4)nc-2c2ccccc21] | 2-(1-Adamantylmethyl)-5-pentyl-2H-pyrazolo[4,3- c[quinolin-3(5)-one; |
| 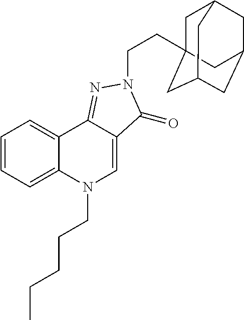 [SMILES: CCCCCn1cc2c(=O)n(CCC34CC5CC(CC(C5)C3)C4)nc-2c2ccccc21] | 2-(1-Adamantylethyl)-5-pentyl-2H-pyrazolo[4,3-c[quinolin- 3(5H)-one; |
| 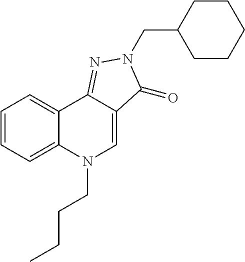 [SMILES: CCCCn1cc2c(=O)n(CC3CCCCC3)nc-2c2ccccc21] | 2-(Cyclohexylmethyl)-5-butyl-2H-pyrazolo[4,3-c]quinolin- 3(5H)-one; |
| 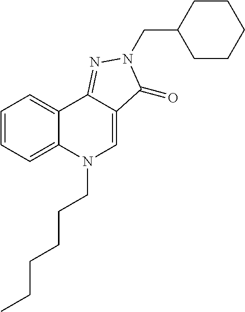 [SMILES: CCCCCCn1cc2c(=O)n(CC3CCCCC3)nc-2c2ccccc21] | 2-(Cyclohexylmethyl)-5-hexyl-2H-pyrazolo[4,3-c]quinolin- 3(5H)-one; |
| 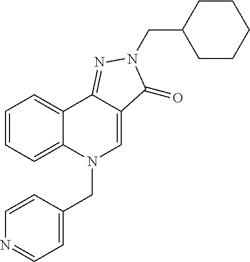 [SMILES: O=c1c2cn(Cc3ccncc3)c3ccccc3c-2nn1CC1CCCCC1] | 2-(Cyclohexylmethyl)-5-(tetrahydro-2H-4-pyranylmethyl)- pyrazolo[4,3-c]quinolin-3(5H)-one; |
| 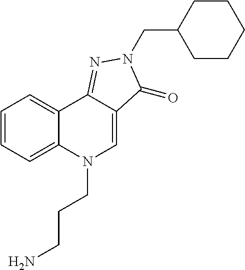 [SMILES: NCCCn1cc2c(=O)n(CC3CCCCC3)nc-2c2ccccc21] | 2-(Cyclohexylmethyl)-5-(4,4,4-trifluroobutyl)-2H- pyrazolo[4,3-c]quinolin-3(5H)-one. |

The compounds of Formula I can be prepared in accordance with the reactions known to persons skilled in the art. The reaction diagrams described in the “Examples” part illustrate possible synthesis approaches.

In a second aspect, the invention relates to the use of the compounds of the invention or the pharmaceutically acceptable solvates thereof as agonists of CB₂.

The compounds of the invention are thus useful in the treatment and/or prevention of illnesses or diseases associated with a change (increase or decrease) in the activity of the CB₂ receptor. The invention therefore also relates to the compounds according to the invention for use as a drug, in particular for use in the treatment and/or prevention of illnesses or diseases mediated by CB₂.

These illnesses or diseases comprise auto-immune diseases, neurodegenerative diseases, inflammatory diseases, osteoporosis, pain and cancers of inflammatory origin. In a particular embodiment, the illnesses or diseases are chosen from chronic inflammatory bowel diseases (CIBD), multiple sclerosis (MS), erythematous lupus, auto-immune thyroiditis, rheumatoid polyarthritis, ankylosing spondylarthritis, atopic dermatitis, hepatitis, Gougerot-Sjögren syndrome, Alzheimer's disease, amyotrophic lateral sclerosis (ALS, also known as Charcot's disease), osteoporosis and pain. Chronic inflammatory bowel diseases (CIBD) for its part include Crohn's disease and haemorrhagic rectocolitis (HRC).

In a preferred embodiment, the invention relates to compounds of Formula I as described above for use in the treatment of chronic inflammatory bowel diseases (CIBD), more particularly Crohn's disease and/or haemorrhagic rectocolitis (HRC).

The present invention, according to another of its aspects, also relates to a method of treating the illnesses and diseases indicated above, comprising the administration to a patient of an effective amount of a compound according to the invention or one of the pharmaceutically acceptable solvates thereof. Preferably, the patient is a warm-blooded animal, more preferentially a human.

According to another aspect, the invention relates to a method of modulating the activity of the CB₂ receptor in a patient, preferably a warm-blooded animal, more preferentially a human, and having need thereof, said method comprising the administration to this patient of an effective amount of a compound according to the invention, or one of the pharmaceutically acceptable solvates thereof.

The invention also relates to a pharmaceutical composition comprising at least one compound of Formula I or at least one pharmaceutically acceptable solvate of said compound and a pharmaceutically acceptable excipient. Said excipients are chosen according to the pharmaceutical form and administration mode required, among the normal excipients that are known to persons skilled in the art.

The pharmaceutical composition of the present invention can be chosen from the pharmaceutical compositions for oral, sublingual, subcutaneous, intramuscular, intravenous, topical, local, intratracheal, intranasal, transdermal or rectal administration. In these compositions, the active principle of Formula I above, or the pharmaceutically acceptable solvate thereof, can be administered in unit administration form, in a mixture with conventional pharmaceutical excipients, to animals or humans for the treatment and/or prevention of the illnesses or diseases mentioned above. The suitable unit administration forms comprise forms by oral pathway such as tablets, soft or hard capsules, powders, granules and oral solutions or suspensions, the sublingual, buccal, intratracheal, intraocular, intranasal and inhalation administration forms, topical, transdermal, subcutaneous, intramuscular or intravenous administration forms, rectal administration forms and implants. For topical application, it is possible to use the compounds according to the invention in creams, gels, ointments or lotions. In a preferred embodiment, it is a case of a pharmaceutical composition for oral administration. Such suitable administration forms, which may be in solid, semi-solid or liquid form according to the administration method, are generally known to persons skilled in the art, reference being made to the last edition of the work “Remington's Pharmaceutical Sciences”.

In a particularly advantageous embodiment, the pharmaceutical composition according to the invention is a pharmaceutical composition for oral administration. The compounds of the invention are in fact, to the knowledge of the inventors, the first agonists of CB₂ that are active by oral pathway in the context of the treatment of chronic inflammatory bowel diseases (CIBD).

### DEFINITIONS

The following definitions and explanation relate to the terms and expressions as used in the present application, comprising the description and the claims.

For the description of the compounds of the invention, the terms and expressions used must, unless indicated to the contrary, be interpreted in accordance with the following definitions.

The term “halo”, alone or as part of another group, designates fluoro, chloro, bromo or iodo. The preferred halo groups are chloro and fluoro, fluoro being particularly preferred.

The term “alkyl”, alone or as part of another group, designates a hydrocarbon radical of formula CₙH₂ₙ₊₁ in which n is an integer number greater or equal to 1.

The term “haloalkyl”, alone or as part of another group, designates an alkyl radical as defined above in which one or more hydrogen atoms are replaced by a halo group as defined above. Preferred linear C4 to C6 haloalkyl radicals are 1,1,1-trifluoro-n-butyl, 1,1,1-trifluoro-n-pentyl and 1,1,1-trifluoro-n-hexyl, 1,1,1-trifluoro-n-butyl being particularly preferred.

The term “cycloalkyl”, alone or as part of another group, designates a saturated mono-, di- or tri-cyclic hydrocarbon radical having 3 to 12 carbon atoms, in particular 5 to 10 carbon atoms, more particularly 6 to 10 carbon atoms. Suitable cycloalkyl radicals comprise, without being limited thereto, cyclopropyl, cyclobutyl, cyclopentyl, cyclohexyl, cycloheptyl, cyclooctyl, norbornyl, adamantyl, in particular adamant-1-yl and adamant-2-yl and 1-decalinyl. Preferred cycloalkyl groups comprise cyclopropyl, cyclohexyl, adamant-1-yl an adamant-2-yl.

The compounds of Formula I may exist in the form of solvates, namely in the form of associations and combinations with one or more solvent molecules, such as for example ethanol or water. When the solvent is water, the term “hydrate” is used.

All the references to compounds of Formula I also designate the solvents thereof.

The compounds of the invention are the compounds of Formula I and the solvates thereof as defined above, including all their polymorphs and crystalline forms, their prodrugs and the compounds or solvates carrying an isotopic label.

The term “patient” designates a warm-blooded animal, preferably a human, awaiting or receiving medical treatment.

The term “human” designates subjects of both sexes and at any stage of development (that is to say neonatal, infant, juvenile, adolescent and adult). In one embodiment, it is a case of an adolescent or an adult, preferably an adult.

The terms “treat” and “treatment” must be understood in their general meaning and thus comprise the improvement and the abrogation of a pathological state.

The terms “prevent” and “prevention” designate the fact of preventing or delaying the appearance of an illness or disease and related symptoms, as well as excluding a patient from developing an illness or disease or reducing the risk of a patient developing an illness or disease.

The term “therapeutically effective amount” or “effective amount” designates the amount of active principle (composed of Formula I) that is sufficient to achieve the desired therapeutic or prophylactic result in the patient to whom it is administered.

The term “pharmaceutically acceptable” designates that a compound or component is not harmful for the patient and that, in the context of a pharmaceutical composition, it is compatible with the other components.

The term “agonist” designates a ligand that activates an intracellular response when it binds to a receptor and covers full agonists as well as partial agonists.

The present invention will be understood better with reference to the following examples. These examples represent certain embodiments of the invention and in no case limit the scope of the invention. The figures serve to illustrate the experimental results.

### FIGURES

FIG. 1A: Survival rate in an acute colitis model in mice for compound 26 in comparison with an agonist of CB₂ of the prior art.

FIG. 1B: Evaluation of the loss of body weight in an acute colitis model in mice for compound 26 in comparison with an agonist of CB₂ of the prior art.

FIGS. 2A-D: Macroscopic score (A), histological damage (B), level of TNFα and IL-1β (C, D) in the colon after colitis caused by TNBS and treatment with compound 26 or an agonist of CB₂ of the prior art.

### EXAMPLES

### Analysis Equipment and Techniques

The purity of the synthesis products was checked by thin-layer chromatography on 60F254 silica gel plates with a thickness of 0.2 mm (ref. 5735 Merck) (revelation: UV (254 and 366 nm) for the products with conjugate bonds, ninhydrin for the amines, iodine in all cases, Dragendorff reagent for the compounds comprising a heterocyclic nitrogen atom). Purifications by column chromatography were carried out on silica gel 60, granulometry 0.040-0.063 mm (ref. 9385.5000 Merck). The eluent was chosen so as to obtain an Rf of between 0.20 and 0.25 on CCM plates. The melting points (Mp) were determined by means of a Büchi SMP 20 apparatus and are not corrected. They are expressed in degrees Celsius (° C.). The infrared spectra were produced on a Bruker Vector 22 Fourier transform spectrometer. The characteristic signals are marked by their wave number expressed in cm⁻¹. ¹H NMR spectra were recorded at 300 MHz on a Bruker AC 300P Fourier transform apparatus, with TMS (trimethylsilane) as the internal reference. This signal is marked by its chemical displacement (δ in ppm), its intensity (H number), its multiplicity (s, singlet; d, doublet; t, triplet; q, quadruplet; m, massive or multiplet) and optionally its coupling constant (J in hertz). In the case of massives, this is not measurable. All the compounds were characterised by LC-MS. The high-performance liquid chromatograph (ODS column, mobile phase: water/acetonitrile/formic acid in gradient mode) is coupled to a UV detector and to a mass detector of the APCI⁺ (atmospheric pressure chemical ionisation) type. The spectra were recorded on an MSQ thermo electron surveyor apparatus.

### Chemical Synthesis

A. Synthesis of the Final Compounds 16 and 21-27 and Comparative Compounds 8, 19

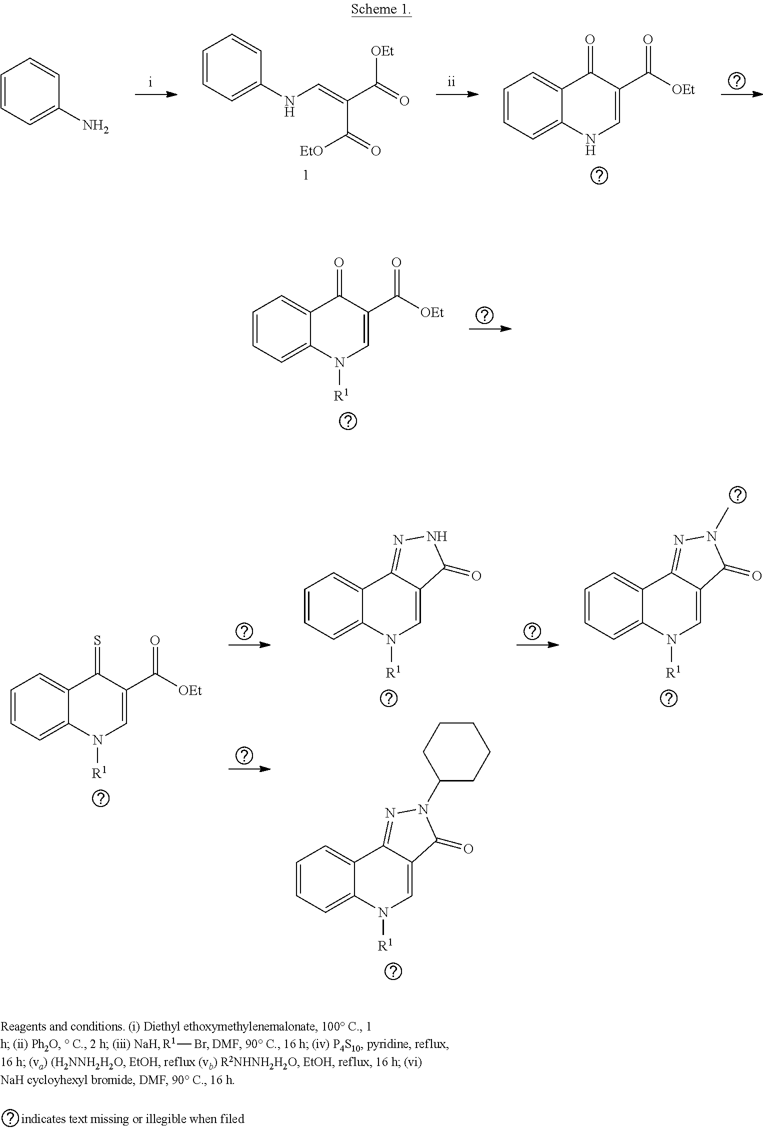

Diethyl 2-((phenylamino)methylene)malonate (1), ethyl 4-oxo-1,4-dihydroquinoline-3-carboxylate (2) and ethyl 4-oxo-1-pentyl-1,4-dihydroquinoline-3-carboxylate (4) (R¹=n-pentyl) have already been described in a previous article (Stern, E., Muccioli, G. G.; Millet, R.; Goossens, J. F.; Farce, A.; Chavatte, P.; Poupaert, J. H.; Lambert, D. M.; Depreux, P.; Hénichart, J. P. Novel 4-Oxo-1,4-dihydroquinoline-3-carboxamide Derivatives as New CB₂ Cannabinoid Receptors Agonists: Synthesis, Pharmacological Properties and Molecular Modeling. J. Med. Chem. 2006, 49, 70-79); the thionation reactions involved in the preparation of the compound (6) have also been described (Stern, E.; Muccioli, G. G.; Bosier, B.; Hamtiaux, L.; Millet, R.; Poupaert, J. H.; Hénichart, J. P.; Depreux, P.; Goossens, J. F.; Lambert, D. M. Pharmacomodulations Around the 4-Oxo-1,4-dihydroquinoline-3-carboxamides, a Class of Potent CB2-Selective Cannabinoid Receptor Ligands: Consequences in Receptor Affinity and Functionality. J. Med. Chem. 2007, 50, 5471-5484).

### General Procedure for Preparing 2H-pyrazolo[4,3-c]quinolin-3(5H)-ones (8 and 16) from 4-thioxoquinoline (6)

Compound 6 (1 equiv.) and hydrazine monohydrate or monosubstituted hydrazine (3 equiv.) in absolute ethanol are refluxed for 14 hours. Di-iso-propylethylamine (DIPEA) (3.2 equiv.) is also added in the case where the monosubstituted hydrazines are in hydrochloric form. After return to ambient temperature, the solvent is evaporated and the residue is shared by a dichloromethane-H₂O mixture. The organic phase is washed with a solution of water saturated with NaCl, dried on MgSO₄ and evaporated in order to give a yellow oil that is purified by silica gel chromatography (CH₂Cl₂/MeOH 95:5, v/v). The yellow solids obtained are recrystallised in acetonitrile.

5-Pentyl-2H-pyrazolo[4,3-c]quinolin-3(5H)-one (8). Yield: 90%. mp>250° C. IR (cm⁻¹) 1610. ¹H NMR (DMSO-d₆) δ 11.42 (s. 1H), 8.67 (s, 1H), 8.14 (d, 1H, J=7.9 Hz), 7.82 (d, 1H, J=8.4 Hz), 7.66 (t, 1H, J=7.3 Hz), 7.52 (t, 1H, J=7.6 Hz), 4.38 (t, 2H, J=7.3 Hz), 1.82-1.68 (m, 2H), 1.38-1.25 (m, 4H), 0.84 (t, 3H, J=6.4 Hz). LC-MS (APCI⁺) m/z, 256.2 (MH⁺).

2-Cyclohexyl-5-pentyl-2H-pyrazolo[4,3-c]quinolin-3 (5H)-one (16). Yield: 77%. mp>250° C. IR (cm⁻¹) 1634. ¹H NMR (CDCl₃) δ 8.43 (d, 1H, J=7.6 Hz), 8.39 (s, 1H), 7.63-7.48 (m, 3H), 4.24 (t, 2H, J=7.2 Hz), 1.86-1.19 (m, 17H), 1.00 (t, 3H, J=6.9 Hz). LC-MS (APCI⁺) m/z 338.2 (MH⁺).

### General Procedure for Preparing 2H-pyrazolo[4,3-c]quinolin-3(5H)-ones (19, 21-27) from Compound (8)

NaH (1.5 equiv., 60% in mineral oil) is added in portions, at 0° C., to a solution containing compound 8 (1 equiv.) in anhydrous DMF (20 ml for 1.2 mmol of 8 or 9). After 30 minutes, the appropriate halogenated derivative (1.5 equiv.) is added and the mixture is heated to 90° C. for 14 hours. After return to ambient temperature, the reaction medium is concentrated under vacuum and the residue is taken up in water and extracted with CH₂Cl₂. The oil obtained is next purified by silica gel chromatography (CH₂Cl₂/MeOH 95:5, v/v) and then recrystallised in acetonitrile.

2-Benzyl-5-pentyl-2H-pyrazolo[4,3-c]quinolin-3(5H)-one (19). Yield: 74%. mp>250° C. IR (cm⁻¹) 1644. ¹H NMR (CDCl₃) δ 8.39 (s, 1H), 8.36 (d, 1H, J=7.6 Hz), 7.72 (t, 1H, J=8.1 Hz), 7.64 (d, 1H, J=8.4 Hz), 7.58-6.99 (m, 6H), 5.26 (s, 2H), 4.26 (t, 2H, J=7.3 Hz), 1.91-1.62 (m, 2H), 1.49-1.27 (m, 4H), 1.00 (t, 3H, J=6.4 Hz). LC-MS (APCI⁺) m/z 346.2 (MH⁺).

2,5-Dipentyl-2H-pyrazolo[4,3-c]quinolin-3(5H)-one (21). Yield: 77%. mp 154° C. IR (cm⁻¹) 1630. ¹H NMR (CDCl₃) δ 8.41 (dd, 1H, J =7.6 Hz, J=1.5 Hz), 8.25 (s, 1H), 7.59-7.51 (m, 3H), 4.24 (t, 2H, J=7.3 Hz), 4.06 (t, 2H, J=7.3 Hz), 1.91 (m, 4H), 1.38 (m, 8H), 0.90 (m, 6H). LC-MS (APCI⁺) m/z 326.2 (MH⁺).

2-Hexyl-5-pentyl-2H-pyrazolo[4,3-c]quinolin-3(5H)-one (22). Yield: 73%. mp 157° C. IR (cm⁻¹) 1632. ¹H NMR (CDCl₃) δ 8.40 (dd, 1H, J=7.9 Hz, J=1.2 Hz), 8.25 (s, 1H), 7.58-7.51 (m, 3H), 4.24 (t, 2H, J=7.3 Hz), 4.06 (t, 2H, J=7.3 Hz), 1.91 (m, 4H), 1.38 (m, 10H), 0.90 (m, 6H). LC-NS (APCI⁺) m/z 340.3 (MH⁺).

2-(Cyclopropylmethyl)-5-pentyl-2H-pyrazolo[4,3-c]quinolin-3(5H)-one (23). Yield: 47%. mp 187° C. IR (cm⁻¹) 1632. ¹H NMR (CDCl₃) δ 8.41 (dd, 1H, J=7.6 Hz), 8.25 (s, 1H), 7.58-7.48 (m, 3H), 4.24 (t, 2H, J=7.3 Hz), 3.89 (d, 2H, J=7.3 Hz), 1.91 (m, 2H), 1.39 (m, 5H), 0.92 (t, 3H, J=7.0 Hz), 0.47 (m, 4H). LC-MS (APCI⁺) m/z 310.1 (MH⁺).

2-(Cyclohexylmethyl)-5-pentyl-2H-pyrazolo[4,3-c]quinolin-3(5H)-one (24). Yield: 37%. mp 200° C. IR (cm⁻¹) 1631. ¹H NMR (CDCl₃) δ 8.41 (d, 1H, J=7.6 Hz), 8.38 (s, 1H), 7.63-7.48 (m, 3H), 4.24 (t, 2H, J=7.3 Hz), 3.88 (d, 2H, J=7.3 Hz), 1.91-1.19 (m, 17H), 0.92 (t, 3H, J=7.0 Hz). LC-MS (APCI⁺) m/z 352.2 (MH⁺).

2-(2-Cyclohexylethyl)-5-pentyl-2H-pyrazolo[4,3-c]quinolin-3(5H)-one (25). Yield: 57%. mp 160° C. IR (cm⁻¹) 1621. ¹H NMR (CDCl₃) δ 8.41 (d, 1H, J=7.9 Hz), 8.24 (s, 1H), 7.60-7.48 (m, 3H), 4.24 (t, 2H, J=7.3 Hz), 4.06 (t, 2H, J=7.3 Hz), 1.91-1.19 (m, 17H), 0.92 (m, 5H). LC-MS (APCI⁺) m/z 366.2 (MH⁺).

2-(1-Adamantylmethyl)-5-pentyl-2H-pyrazolo[4,3-c]quinolin-3(5H)-one (26). Yield: 32%. mp 225° C. IR (cm⁻¹) 1629. ¹H MNR (CDCl₃) δ 8.39 (d, 1H, J=7.6 Hz), 8.36 (s, 1H), 7.63-7.47 (m, 3H), 4.23 (t, 2H, J=7.3 Hz), 3.73 (s, 2H), 1.97-1.39 (m, 21H), 0.93 (t, 3H, J=7.0 Hz). LC-MS (APCI⁺) m/z 404.2 (MH⁺).

2-(1-Adamantylethyl)-5-pentyl-2H-pyrazolo[4,3-c]quinolin-3 (5H)-one (27). Yield: 35%. mp 237° C. IR (cm⁻¹) 1625. ¹H NMR (CDCl₃) δ 8.40 (d, 1H, J=7.9 Hz), 8.26 (s, 1H), 7.63-7.47 (m, 3H), 4.24 (t, 2H, J=7.2 Hz), 4.06 (m, 2H), 3.73 (s, 2H), 2.01-1.33 (m, 23H), 0.93 (t, 3H, J=7.0 Hz). LC-MS (APCI⁺) m/z 418.2 (MH⁺).

### B. Synthesis of 2-(cyclohexylmethyl)-5-alkyl-pyrazolo[4,3-c]quinolin-3(5H)-ones (Final Compounds 40-43)

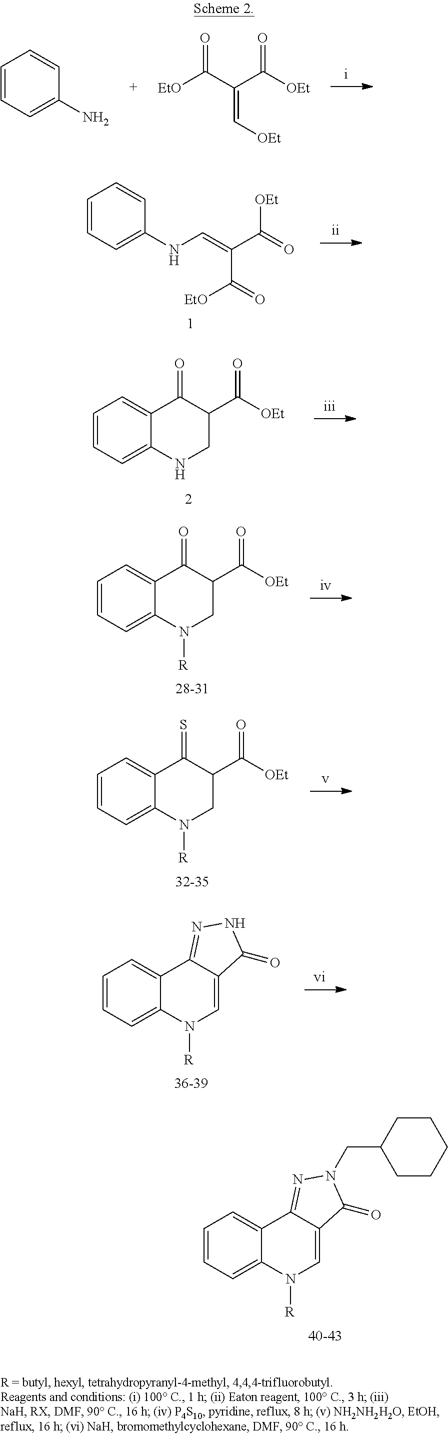

Diethyl 2-((phenylamino)methylene)malonate (1) and ethyl 4-oxo-1,4-dihydroquinoline-3-carboxylate (2) have been described in a previous article (Stern, E., Muccioli, G. G.; Millet, R.; Goossens, J. F.; Farce, A.; Chavatte, P.; Poupaert, J. H.; Lambert, D. M.; Depreux, P.; Hénichart, J. P. Novel 4-Oxo-1,4-dihydroquinoline-3-carboxamide Derivatives as New CB₂ Cannabinoid Receptors Agonists: Synthesis, Pharmacological Properties and Molecular Modeling. J. Med. Chem. 2006, 49, 70-79).

### General Procedure for Preparing Ethyl 1-alkyl-4-oxo-1,4-dihydroquinoline-3-carboxylates (28-31)

In a 100 ml two-neck flask, 3 g of quinoline 2 is solubilised in 15 ml of anhydrous DMF. The medium is placed in an ice bath and put under nitrogen. Sodium hydride (0.607 g; 15.18 mmol; 60% in mineral oil) is added in fractions. The mixture is left under agitation for 15 minutes at 0° C. The halogenated derivative (20.70 mmol) is then added and the reaction is heated to 90° C. for 16 hours. The medium is poured into 150 ml of iced distilled water: if the alkylated derivative obtained is solid, it is then drained, washed with water, dried and then recrystallised. Otherwise it is extracted with dichloromethane, washed with water, dried by rotary evaporation and then purified on silica column.

Ethyl 1-butyl-4-oxo-1,4-dihydroquinoline-3-carboxylate (28). Recrystallisation Solvent: ethyl acetate. Yield: 81%. mp =123° C. IR (cm⁻¹) 1642, 1600. ¹H NMR (CDCl₃) δ (ppm) 8.56 (d, 1H, J=8.5 Hz), 8.49 (s, 1H), 7.69 (t, 1H, J=8.4 Hz), 7.47-7.40 (m, 2H), 4.42 (q, 2H, J=7.1 Hz), 4.19 (t, 1H, J=7.4 Hz), 1.92-1.88 (q, 2H), 1.50-1.41 (m, 5H), 1.01 (t, 3H, J=7.3 Hz). LC-MS (ESI⁺) m/z 270.12 (MH⁺).

Ethyl 1-hexyl-4-oxo-1,4-dihydroquinoline-3-carboxylate (29). Crystallisation Solvent: cyclohexane. Yield: 96%. mp=67° C. IR (cm⁻¹) 1710, 1606. ¹H NMR (CDCl₃) δ (ppm) 8.59-8.53 (m, 2H), 8.49 (s, 1H), 7.70 (t, 1H, J=8.2 Hz), 7.49-7.43 (m, 2H), 4.42 (q, 1H, J=7.0 Hz), 4.21 (t, 2H, J=7.4 Hz), 1.93-1.89 (m, 2H), 1.46-1.26 (m, 9H), 0.90 (t, 3H, J=6.8 Hz). LC-MS (ESI⁺) m/z 338.16 (MH⁺).

Ethyl 1-(tetrahydro-2H-4-pyranylmethyl)-4-oxo-1,4-dihydroquinoline-3-carboxylate (30). Chromatography eluent: dichloromethane/methanol 9:1, v/v. Yield: 58%. mp=103° C. IR (cm⁻¹) 1714, 1607, 1087. ¹H NMR (CDCl₃) δ (ppm) 8.56 (d, 1H, J=8.0 Hz), 8.43 (s, 1H), 7.71 (t, 1H, J=8.6 Hz), 7.49-7.41 (m, 2H), 4.41 (q. 2H, J=7.1 Hz), 4.07 (d, 2H, J=7.3 Hz), 4.03-4.99 (dd, 2H, J₁=11.3 Hz, J₂=3.2 Hz), 3.33 (Td, 2H, J₁=11.6 Hz, J₂=2.3 Hz), 2.23-2.15 (m, 1H), 1.57-1.49 (m, 4H), 1.43 (t, 3H, J=7.1 Hz). LC-MS (ESI⁺) m/z 316.12 (MH⁺).

Ethyl 1-4,4,4-trifluorobutyl-4-oxo-1,4-dihydroquinoline-3-carboxylate (31). Chromatography eluent: dichloromethane/ethyl acetate 7:3, v/v. Yield: 23%. mp=132° C. IR (cm⁻¹) 1670, 1641, 1607. ¹H NMR (CDCl₃) δ (ppm) 8.55 (d, 1H, J=8.07 Hz), 8.49 (s, 1H), 7.71 (t, 1H, J=7.17 Hz), 7.50-7.40 (m, 2H), 4.40 (q. 2H, J=7.11 Hz), 4.29 (t, 2H, J=7.56 Hz), 2.29-2.18 (m, 2H), 1.43 (t, 3F, J=7.11 Hz). LC-MS (APCI⁺) m/z 296.11 (MH⁺).

### General Procedure for Preparing Ethyl 1-alkyl-4-thioxo-1,4-dihydroquinoline-3-carboxylates (32-35)

In a round-bottom flask, ethyl 1-alkyl-4-oxo-1,4-dihydroquinoline-3-carboxylate is solubilised in pyridine, and then phosphorus pentasulfide is added. The mixture is refluxed for 8 hours. After cooling to ambient temperature, the reaction medium is poured into distilled water. The sulfurated product is extracted with ethyl acetate. The organic phase is washed with a 1N hydrochloric acid solution, then with water, dried on magnesium sulfate, filtered and dried under reduced pressure. The red oil obtained is purified by silica gel chromatography.

Ethyl 1-butyl-4-thioxo-1,4-dihydroquinoline-3-carboxylate (32). Chromatography Eluent: dichloromethane/ethyl acetate 1:1, v/v. Yield: 82%. mp=116° C. IR (cm⁻¹) 1718, 1596. ¹H NMR (CDCl₃) δ (ppm) 8.92 (s, 1H), 8.58 (d, 1H, J=7.7 Hz), 7.85 (t, 1H, J=8.0 Hz), 7.67 (d, 1H, J=8.7 Hz), 7.59 (t, 1H, J=7.6 Hz), 4.50-4.48 (m, 4H), 1.93-1.92 (m, 2H), 1.50-1.47 (m, 5H), 1.43 (t, 3H, J=6.90 Hz). LC-MS (ESI⁺) m/z 290.12 (MH⁺).

Ethyl 1-hexyl-4-thioxo-1,4-dihydroquinoline-3-carboxylate (33). Chromatography Eluent: dichloromethane/ethyl acetate 1:1, v/v. Yield: 85%. mp=92° C. IR (cm⁻¹) 1720, 1598. ¹H NMR (CDCl₃) δ (ppm) 9.12 (d, 1H, J=8.5 Hz), 7.90 (s, 1H), 7.72 (t, 1H, J=8.2 Hz), 7.52-7.46 (m, 2H), 4.43 (q, 2H, J=7.1 Hz), 4.22 (t, 2H, J=6.9 Hz), 1.93 (q, 2H, J=7.2 Hz), 1.40 (t, 3H, J=6.2 Hz), 1.37-1.26 (m, 6H), 0.91 (t, 3H, J=6.80 Hz). LC-MS (ESI⁺) m/z 318.16 (MH⁺).

Ethyl 1-(tetrahydro-2H-4-pyranylmethyl)-4-thioxo-1,4-dihydroquinoline-3-carboxylate (34). Chromatography eluent: dichloromethane/ethyl acetate 1:1, v/v. Yield: 71%. mp =140° C. IR (cm⁻¹) 1717, 1596, 1014. ¹H NMR (CDCl₃) δ (ppm) 9.43 (d, 1H, J=8.0 Hz), 7.94 (s, 1H), 7.74 (t, 1H, J=7.0 Hz), 7.54-7.48 (m, 2H), 4.43 (q, 2H, J=7.1 Hz), 4.12 (d, 2H, J=7.1 Hz), 4.02 (dd, 2H, J₁=7.3 Hz, J₂=3.1 Hz), 3.34 (Td, 2H, J₁=11.8 Hz, J₂=2.2 Hz), 2.21-2.17 (m, 1H), 1.55-1.45 (m, 4H), 1.41 (t, 3H, J=7.2 Hz). LC-MS (ESI⁺) m/z 332.08 (MH⁺).

Ethyl 1-(4,4,4-trifluorobutyl)-4-thioxo-1,4-dihydroquinoline-3-carboxylate (35). Chromatography eluent: dichloromethane/ethyl acetate 1:1, v/v. Yield: 89%. mp=85° C. IR (cm⁻¹) 1718. ¹H NMR (CDCl₃) δ (ppm) 8.55 (d, 1H, J=8.07 Hz), 8.49 (s, 1H), 7.91 (t, 1H, J=7.17 Hz), 7.63-7.50 (m, 2H), 4.52 (q, 2H, J=7.11 Hz), 4.29 (t, 2H, J=7.56 Hz), 2.41-2.31 (m, 2H), 1.53 (t, 3F, J=7.11 Hz). LC-MS (APCI⁺) m/z 296.11 (MH⁺).

### General Procedure for Preparing 5-alkyl-2H-pyrazolo[4,3-c]quinolin-3(5H)-ones (36-39)

In a 100 ml round-bottom flask, ethyl 1-alkyl-4-thioxoquinoline-3-carboxylate is solubilised in absolute ethanol. Hydrazine monohydrate is added and the medium is refluxed for 16 hours. After the solvent has been evaporated under reduced pressure, the residue obtained is taken up in distilled water, dewatered, dried and recrystallised in absolute ethanol.

5-Butyl-2H-pyrazolo[4,3-c]quinolin-3(5H)-one (36). Yield: 87%. mp>250° C. IR (cm⁻¹) 1627. ¹H NMR (DMSO-d₆) δ (ppm) 11.37 (s, 1H), 8.64 (s, 1H), 8.14 (d, 2H, J=7.8 Hz), 7.82 (d, 1H, J=8.6 Hz), 7.66 (t, 1H, J=7.0 Hz), 7.53 (t, 1H, J=7.3 Hz), 4.39 (t, 2H, J=7.2 Hz), 1.75 (quint, 2H), 1.33 (sext, 2H), 0.92 (t, 3H, J=6.30 Hz). LC-MS (ESI⁺) m/z 242.14 (MH⁺).

5-Hexyl-2H-pyrazolo[4,3-c]quinolin-3(5H)-one (37). Yield: 85%. mp=242° C. IR (cm⁻¹) 1607. ¹H NMR (CDCl₃) δ (ppm) 9.42 (s, 1H), 8.35 (d, 1H, J=8.3 Hz), 8.32 (s, 1H), 7.63 (t, 1H, J=9.0 Hz), 7.53 (m, 2H), 4.26 (t, 2H, J=7.3 Hz), 1.87 (m, 4H), 1.43-1.25 (m, 4H), 0.89 (t, 3H, J=6.80 Hz). LC-MS (ESI⁺) m/z 270.12 (MH⁺).

5-(Tetrahydro-2H-4-pyranylmethyl)-2H-pyrazolo[4,3-c]quinolin-3(5H)-one (38). Yield: 82%. mp >250° C. IR (cm⁻¹) 1607, 1083. ¹H NMR (CDCl₃) δ (ppm) 9.00 (s, 1H), 8.37 (d, 1H, J=7.8 Hz), 8.21 (s, 1H), 7.69-7.47 (m, 3H), 4.14 (d, 2H, J=7.2 Hz), 4.02 (Dd, 2H, J₁=12.0 Hz, J₂=4.0 Hz), 3.34 (Td, 2H, J₁=11.6 Hz, J₂=2.3 Hz), 2.22 (m, 1H), 1.63-1.46 (m, 4H). LC-MS (ESI⁺) m/z 284.13 (MH⁺).

5-(4,4,4-Trifluorobutyl)-2H-pyrazolo[4,3-c]quinolin-3(5H)-one (39). Yield: 72%. mp >250° C. IR (cm⁻¹) 1627. ¹H NMR (DMSO-d₆) δ (ppm) 11.39 (s, 1H), 8.65 (s, 1H), 8.05 (d, 1H, J=7.4 Hz), 8.86 (d, 1H, J=8.3 Hz), 7.67 (t, 1H, J=7.3 Hz), 7.52 (t, 1H, J=7.5 Hz), 4.45 (t, 2H, J=7.3 Hz), 2.45-2.36 (m, 2H), 2.02-1.99 (m, 2H), 1.40 (t, 3F, J=7.11 Hz). LC-MS (APCI⁺) m/z 321.30 (MH⁺).

### General Procedure for Preparing 2-(cyclohexylmethyl)-5-alkyl-2H-pyrazolo[4,3-c]quinolin-3(5H)-ones (40-43)

In a two-neck flask, 5-alkyl-2H-pyrazolo[4,3-c]quinolin-3(5H)-one is solubilised in anhydrous DMF. The medium is placed under nitrogen at 0° C. and sodium hydride is added by fractions. The medium is then left under agitation for 30 minutes and then the brominated derivative is added. The reaction is left under agitation at 90° C. for 16 hours. The reaction is poured into distilled water. The precipitate is dewatered, washed with water, dried and then purified by silica gel chromatography followed by recrystallisation in acetonitrile.

2-(Cyclohexylmethyl)-5-butyl-2H-pyrazolo[4,3-c]quinolin-3(5H)-one (40). Chromatography eluent: ethyl acetate/ethanol 9:1, v/v. Yield: 30%. mp=216° C. IR (cm⁻¹) 1628. ¹H NMR (DMSO-d₆) δ (ppm) 8.40 (d, 1H, J=7.7 Hz), 8.24 (s, 1H), 7.64-7.49 (m, 3H), 4.26 (t, 2H, J=6.9 Hz), 3.88 (d, 2H, J=7.2 Hz), 1.93-1.10 (m, 15H), 1.00 (t, 3H, J=7.40 Hz). LC-MS (ESI⁺) m/z 338.16 (MH⁺).

2-(Cyclohexylmethyl)-5-hexyl-2H-pyrazolo[4,3-c]quinolin-3(5H)-one (41). Chromatography eluent: ethyl acetate/ethanol 9:1, v/v. Yield: 57%. mp=193° C. IR (cm⁻¹) 1631. ¹H NMR (DMSO-d₆) δ (ppm) 8.73 (s, 1H), 8.15 (d, 1H, J=7.9 Hz), 7.84 (d, 1H, J=8.7 Hz), 7.67 (t, 1H, J=7.1 Hz), 7.52 (t, 1H, J=7.8 Hz), 4.40 (t, 2H, J=7.0 Hz), 3.68 (d, 2H, J=7.1 Hz), 1.78-0.98 (m, 19H), 0.83 (t, 3H, J=6.9 Hz). LC-MS (ESI⁺) m/z 366.26 (MH⁺).

2-(Cyclohexylmethyl)-5-(tetrahydro-2H-4-pyranylmethyl)-pyrazolo[4,3-c]quinolin-3(5H)-one (42). Chromatography eluent: ethyl acetate/ethanol 9:1, v/v. Yield: 12%. mp >250° C. IR (cm⁻¹) 1627, 1091. ¹H NMR (DMSO-d₆) δ (ppm) 8.69 (s, 1H), 8.15 (d, 1H, J=7.9 Hz), 7.90 (d, 1H, J=8.5 Hz), 7.67 (t, 1H, J=7.3 Hz), 7.53 (t, 1H, J=7.4 Hz), 4.31 (d, 2H, J=7.3 Hz), 3.81 (d, 2H, J=11.2 Hz), 3.67 (d, 2H, J=7.0 Hz), 3.18 (t, 2H, J=9.2 Hz), 2.1-0.9 (m, 16H). LC-MS (ESI⁺) m/z 380.23 (MH⁺).

2-(Cyclohexylmethyl)-5-(4,4,4-trifluorobutyl)-2H-pyrazolo[4,3-c]quinolin-3(5H)-one (43). Chromatography eluent: dichloromethane/methanol 9:1, v/v. Yield: 15%. mp=224° C. IR (cm⁻¹) 1628. ¹H NMR (DMSO-d₆) δ (ppm) 8.71 (s, 1H), 8.15 (d, 1H, J=6.57 Hz), 7.90 (d, 1H, J=8.52 Hz), 7.68 (t, 1H, J=7.14 Hz), 7.53 (t, 1H, J=7.02 Hz), 4.45 (t, 2H, J=7.23 Hz), 3.66 (d, 2H, J=7.02 Hz), 2.07-1.1 (m, 20H). LC-MS (ESI⁺) m/z 392.20 (MH⁺).

### In Vitro Pharmacological Studies

Measurement of Affinity. The binding studies were carried out in accordance with the protocols described by El Bakali et al. (El Bakali, J.; Muccioli, G. G.; Renault, N.; Pradal, D.; Body-Malapel, M.; Djouina, M.; Hamtiaux, L.; Andrzejak, V.; Desreumaux, P.; Chavatte, P.; Lambert, D. M.; Millet, R. 4-Oxo-1,4-Dihydropyridines as Selective CB₂ Cannabinoid Receptor Ligands: Structural Insights into the Design of a Novel Inverse Agonist Series. J. Med. Chem. 2010, 53, 7918-7931) but applying a small modification. Briefly, the [3H]-CP-55,940 (0.5 nM) chosen as a radioligand for the CB₁ and CB₂ human receptors is added to 6 μg of membranes resuspended in 550 μl (final volume) of buffer (20 mM Hepes, 5 mM MgCl₂, 1 mM EDTA, 0.3% bovine serum albumin, pH 7.4). After 1 hour at 30° C., incubation is stopped and the solution is quickly filtered on Harvester, through the “Unifilter 96 (PerkinElmer)” filter previously saturated with bonding buffer (20 mM Hepes, 5 mM MgCl₂, 1 mM EDTA, 0.3% bovine serum albumin, pH 7.4), and then washed 20 times with iced binding buffer without serum albumin. The radioactivity of the filters was measured using the “TopCount NXT Microplate Scintillation Counter (PerkinElmer)” after addition of 60 μl of “MicroScint 40 (PerkinElmer)” scintillation liquid. The experiments were carried out in triplicate. The non-specific binding was determined in the presence of 5 μM (R)-(+)-WIN 55,212-2 (Sigma).

Dosing of [³⁵5]-GTPγS. The studies were carried out at 30° C. in tubes containing 10 μg of protein in 0.5 ml (final volume) of buffer (20 mM Hepes, 10 mM MgCl₂, 100 mM NaCl, 0.1% bovine serum albumin, pH 7.4) supplemented with 30 μm of GDP. The dosing was initiated by the addition of [³⁵S]-GTPγS (0.1 nM, final concentration). After 1 hour at 30° C., the incubation was stopped and the solution was quickly filtered on the “Unifilter-96 GF/B” filter and washed 20 times with iced bonding buffer. The radioactivity on the filters was counted as mentioned above. The non-specific binding was measured in the presence of 100 μm of Gpp(NH)p. The results were expressed in EC₅₀ (nM) and Emax (%).

Analysis of the Data. The values of Ki and EC₅₀ were determined by non-linear regression carried out by the “GraphPad Prism 5.0” program (GraphPad Software, San Diego). The statistical analysis of the results of the [³⁵S]-GTPγS dosing was carried out by means of the “Student” test.

Tables 2 and 3 below summarise the binding affinities for CB1 and CB₂.

TABLE 2
Binding affinities

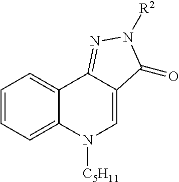

| | | Binding affinity | Binding affinity |
|---|---|---|---|
| | | hCB₂ | hCB₁ |
| Compound | R² | Ki (nM) | Ki (nM) |
| 8 | H | >3000 | >3000 |
| 16 | 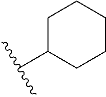 [SMILES: *C1CCCCC1] | 18.8 ± 3.3 | >3000 |
| 19 | 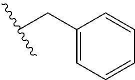 [SMILES: *Cc1ccccc1] | >3000 | >3000 |
| 21 | n-C₅H₁₁ | 4.3 ± 0.3 | >3000 |
| 22 | n-C₆H₁₃ | 7.7 ± 0.3 | >3000 |
| 23 | 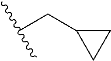 [SMILES: *CC1CC1] | 10.4 ± 3.1 | >3000 |
| 24 | 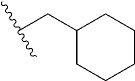 [SMILES: *CC1CCCCC1] | 0.62 ± 0.03 | >3000 |
| 25 | 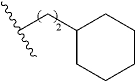 | 8.0 ± 0.4 | >3000 |
| 26 | 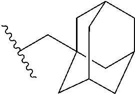 [SMILES: *CC12CC3CC(CC(C3)C1)C2] | 0.39 ± 0.03 | >3000 |
| 27 | 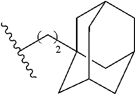 | 26.1 ± 3.8 | >3000 |

TABLE 3
Binding affinities

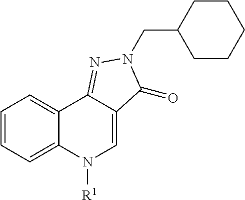

| | | Binding affinity | Binding affinity |
|---|---|---|---|
| | | hCB₂ | hCB₁ |
| Compound | R¹ | Ki (nM) | Ki (nM) |
| 40 | n-C₄H₉ | 14.8 | >10000 |
| 41 | n-C₆H₁₃ | 31.1 | >10000 |
| 42 | 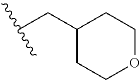 [SMILES: *CC1CCOCC1] | 5.32 | 520 |
| 43 | n-C₃H₆CF₃ | 6.17 | 1680 |

The results obtained for compound 19, which constitutes a comparative example of a compound of Formula I in which R² is an aromatic (benzyl) group, demonstrate that the presence of a non-aromatic hydrophobic aliphatic group in position N−2 (R² group) is crucial for affinity for CB₂. This is because, unlike compound 19, the compounds of the invention, which all have an alkyl, cycloalkyl or cycloalkylalkyl group in position N−2 (R² group), are all active at the nanomolar level.

A comparison of compounds 16, 24 and 25 indicates that, in the case of a cycloalkyl or cycloalkylalkyl group, the choice of a cycloalkylmethyl group gives the best results with regard to affinity for CB₂ while remaining selective for CB₂ vis-à-vis CB₁. This observation is also confirmed for compounds 26 (R²=1-adamantylmethyl) and 27 (R²=1-adamantylethyl).

In addition, in the case of the use of a cycloalkyl or cycloalkylalkyl group in position N−2 (R² group), the size of the ring also appears to impact on the affinity of the compound for CB2. For example, the comparison of compounds 23, 24 and 26 indicates that, the larger the ring (adamantyl>cyclohexyl>cyclopropyl), the better the affinity for CB₂. Linear alkyl chains in position N−2 (R² group) also result in high affinities at a nanomolar level for CB₂.

It is also important to note, and as indicated above, that replacing the alkyl, cycloalkyl or cycloalkylalkyl group in position N−2 (R² group) with an aromatic residue (benzyl group) give rise to a drastic loss in activity (compounds 21, 24, 26 versus 19).

Surprisingly, none of the compounds, with the exception of compounds 42 and 43, has an affinity for CB₁ (hCB₁; Ki>3000), demonstrating that these compounds have a highly selective effect (CB₂ versus CB₁).

The results also emphasise that it is possible to vary the length of the alkyl chain in R¹.

Table 4 below summarises the functional activities of certain compounds of the invention for the cannabinoid receptor hCB₂.

TABLE 4
Functional activities

[³⁵S]-GTPγS (hCB₂)

| Compound | EC₅₀ (nM) | Emax (%) |
|---|---|---|
| 16 | 204 ± 41 | 160 ± 3 |
| 24 | 14.3 ± 4.0 | 149 ± 4 |
| 25 | 5.4 ± 1.1 | 141 ± 3 |
| 26 | 64 ± 10 | 163 ± 4 |

With regard to the functional activities, compounds 16 and 24-26 are all agonists with an EC₅₀ ranging from 5.4 nM to 204 nM and an Emax of around 150%.

### In Vivo Pharmacology

C57B16 male mice (n=10 per group) have free access to food and water. To cause colitis, the mice are anaesthetised for 90-120 minutes by subcutaneous administration of zylasine-ketamine (50 mg/kg) diluted in physiological serum, then receive an intrarectal administration of TNBS (40 μl, 150 mg/kg) diluted in a 1:1 mixture of 0.9% NaCl and 100% ethanol. The novel CB2 agonists are diluted in 0.5% carboxymethyl cellulose (Sigma-Aldrich, Saint Quentin Fallavier, France) for a dose administered by gavage of 0.1, 1 and 10 mg/kg body weight/day. JWH133, a known CB2 agonist (Kimball E S, Schneider C R, Wallace N H, Hornby P J. Agonists of cannabinoid receptor 1 and 2 inhibit experimental colitis induced by oil of mustard and by dextran sulfate soldium. Am. J. Physiol. Gastrointest. Liver Physiol., 2006, 291, G364-71.), is administered intraperitoneally at a dose of 0.1 mg/kg body weight/day. The animals are euthanised 5 days after the administration of TNBS. The intensity of the colitis at macroscopic and histological levels is assessed in each colon blind by 2 investigators. The scale of the macroscopic scores of the lesions ranges from 0 to 10 according to characteristics reflecting inflammation, such as hyperaemia, thickening of the intestinal wall and the extent of ulcerations. A localised colon biopsy precisely 2 cm above the anal channel is used for histological analysis following a May-Grunwald Giemsa colouring. The histological score varies between 0 and 6 and takes into account the degree of inflammatory infiltrate, the presence of erosion, ulcerations and necrosis of the mucosa, and the level of extension of the lesions in depth and on the surface reached. Two other colon biopsies are frozen and used to analyse the levels of mRNA of inflammatory cytokines by PCR in real time. The total mRNAs of the colon are extracted using the Nucleospin RNAII kit (Macherey Nagel, Hoerdt, France) and then retrotranscripts using the High-Capacity cDNA Reverse Transcription Kit (Applied Biosystems), Foster City, USA). The real-time PCR is carried out with SYBR Green (Applied Biosystems, Foster City, USA). The specific primers for TNF-alpha (TNF-alpha F and TNF-alpha R), IL-1 beta (IL1-beta F and IL1-beta R) and POLR2A (POLR2A F and POLR2A R) as reference gene were chosen using the Primer Express 2 software (Applied Biosystems, Foster City, USA). For the graphical representation of the quantitative PCR data, the values of Ct obtained for the target genes are compared with those of the reference gene, using the ΔΔCt method, that is to say: ΔΔCt=(Ct of the target gene−Ct of the reference gene) treated mouse−(Ct of the target gene−Ct of the reference gene) non-treated mouse, and the final data are derived from 2-ΔΔCt.

Compound 26 was evaluated in mice in a model of colitis caused by TNBS in accordance with a described protocol (Desreumaux, P.; Dubuquoy, L.; Nutten, S.; Peuchmaur, M.; Englaro, W.; Schoonjans, K.; Derijard, B.; Desvergne, B.; Wahli, W.; Chambon, P.; Leibowitz, M. D.; Colombel, J. F.; Auwerx, J. Attenuation of Colon Inflammation Through Activators of the Retinoid X Receptor (RXR)/Peroxisome Proliferator-Activated Receptor Gamma (PPARgamma) Heterodimer. A Basis for New Therapeutic Strtegies. J. Exp. Med. 2001, 193, 827-838). Thus compound 26 was administered orally in carboxymethyl cellulose (CMC) each day at a dose of 0.1, 1 and 10 mg/kg for seven days, and two days before the induction of colitis. A control with an administration of CMC alone was effected.

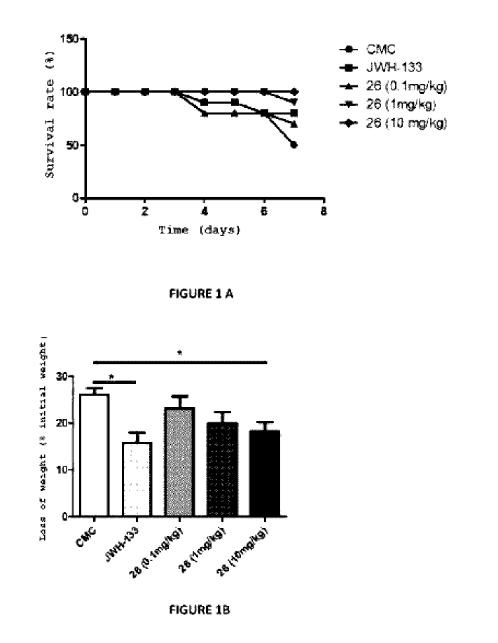

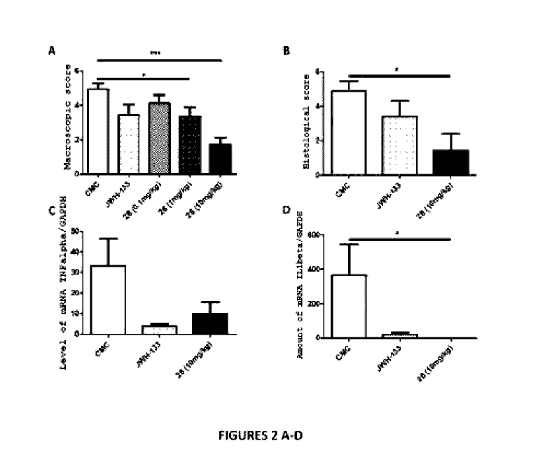

First of all the survival rates throughout the study and the loss of weight before euthanasia of the evaluated mice (five days after the administration of TNBS) were examined. A reduction in mortality for the mice treated with compound 26 compared with the untreated mice (FIG. 1A) was noted. In addition, the daily treatment attenuates in a dose-dependent manner the loss of weight generally observed during colitis induced with TNBS (FIG. 2B).

After the euthanasia of the various groups, the colons of each group were examined and the damage was measured. The results showed that compound 26 reduces the macroscopic scores (FIG. 2A) in a dose-dependent manner with a powerful effect when it is administered orally at a dose of 10 mg/kg (1.7 versus 1.9). This protective effect was confirmed by the histological evaluation. Thus compound 26 attenuates the histological scores by 65% (FIG. 2B). The levels of TNFα and 11-1β, two cytokines largely involved in the inflammatory response, were also quantified. Compound 26.

Thus all the data show that compound 26 is effective in mice against colitis in a dose-dependent manner after oral administration.

1. A compound of Formula I: 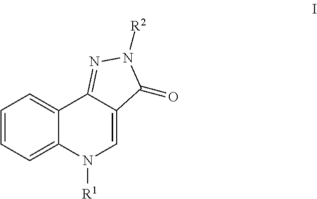
or a pharmaceutically acceptable solvate thereof,
wherein:
R¹ is a linear C4 to C6 alkyl, linear C4 to C6 haloalkyl or C1 to C2 tetrahydropyranylalkyl; and
R² is C3 to C6 alkyl, cycloalkyl or cycloalkylalkyl.

2. The compound or solvate according to claim 1, wherein R¹ is C4 to C6 linear alkyl, C3 to C5 linear trifluoromethylalkyl or C1 to C2 tetrahydropyranylalkyl.

3. The compound or solvate according to claim 1, wherein R² is C5 to C6 alkyl, C5 to C12 cycloalkyl or C3 to C12 cycloalkyl-linear C1 to C3 alkyl.

4. The compound or solvate according to claim 1, wherein R² is selected from the group consisting of n-pentyl, n-hexyl, cyclohexyl, cyclopropylmethyl, cyclohexylmethyl, cyclohexylethyl, adamantylmethyl and adamantylethyl.

5. The compound or solvate according to claim 1, selected from the group consisting of:
2-Cyclohexyl-5-pentyl-2H-pyrazolo[4,3-c]quinolin-3(5R)-one;
2,5-Dipentyl-2H-pyrazolo[4,3-c]quinolin-3(5H)-one;
2-Hexyl-5-pentyl-2H-pyrazolo[4,3-c]quinolin-3(5H)-one;
2-(Cylopropylmethyl)-5-pentyl-2H-pyrazolo[4,3-c]quinolin-3(5H)-one;
2-(Cyclohexylmethyl)-5-pentyl-2H-pyrazolo[4,3-c]quinolin-3(5H)-one;
2-(Cyclohexylethyl)-5-pentyl-2H-pyrazolo[4,3-c]quinolin-3(5H)-one;
2-(1-Adamantylmethyl)-5-pentyl-2H-pyrazolo[4,3-c]quinolin-3(5H)-one;
2-(1-Adamantylethyl)-5-pentyl-2H-pyrazolo[4,3-c]quinolin-3(5H)-one;
2-(Cyclohexylmethyl)-5-butyl-2H-pyrazolo[4,3-c]quinolin-3(5H)-one;
2-(Cyclohexylmethyl)-5-hexyl-2H-pyrazolo[4,3-c]quinolin-3(5H)-one;
2-(Cyclohexylmethyl)-5-(tetrahydro-2H-4-pyranylmethyl)-pyrazolo[4,3-c]quinolin-3(5H)-one; and
2-(Cyclohexylmethyl)-5-(4,4,4-trifluorobutyl)-2H-pyrazolo[4,3-c]quinolin-3(5H)-one.

6. A pharmaceutical composition, comprising at least one compound or a pharmaceutically acceptable solvate thereof according to claim 1 and at least one pharmaceutically acceptable excipient.

7-11. (canceled)

12. A method of treating an illness or disease mediated by CB₂, comprising the step of administering an effective amount of a compound according to claim 1, or a pharmaceutically acceptable solvate thereof, to a patient in need thereof.

13. The method according to claim 12, wherein the illness or disease mediated by CB₂ is selected from the group consisting of auto-immune illnesses, neurodegenerative diseases, inflammatory illnesses, osteoporosis, pain and cancers of inflammatory origin.

14. The method according to claim 12, wherein the illness or disease is selected from the group consisting of chronic inflammatory bowel diseases (CIBD), multiple sclerosis (MS), erythematous lupus, auto-immune thyroiditis, rheumatoid polyarthritis, ankylosing spondylarthritis, atopic dermatitis, hepatitis, Gougerot-Sjögren syndrome, Alzheimer's disease, amyotrophic lateral sclerosis (ALS, also known as Charcot's disease), osteoporosis and pain.

15. The method according to claim 12, wherein the illness or disease is chronic inflammatory bowel disease (CIBD).
